(19) United States
(12) Patent Application Publication
(10) Pub. No.: US 2009/0181997 A1
(43) Pub. Date: Jul. 16, 2009

(54) THERAPEUTIC CANCER TREATMENTS

(76) Inventors: David GRAYZEL, Winchester, MA (US); Robert Ross, Brookline, MA (US); John MacDougall, Hingham, MA (US)

Correspondence Address: MORRISON & FOERSTER LLP, 12531 HIGH BLUFF DRIVE, SUITE 100, SAN DIEGO, CA, 92130-2040, US

(21) Appl. No.: 12/343,245

(22) Filed: Dec. 23, 2008

Related U.S. Application Data
(60) Provisional application No. 61/017,160, filed on Dec. 27, 2007.
(60) Provisional application No. 61/118,969, filed on Dec. 1, 2008.

(51) Int. Cl. A61K 31/438

(52) U.S. Cl. 514/278

(57) ABSTRACT

The invention is directed to use of hedgehog inhibitors in conjunction with chemotherapy in treating cancers, and preventing relapses thereby.

This application claims the benefit of U.S. patent application Ser. No. 11/965,688, filed Dec. 27, 2007, U.S. Provisional Patent Application No. 61/017,160, filed Dec. 27, 2007, and U.S. Provisional Application No. 61/118,969, filed Dec. 1, 2008, each of which is incorporated by reference in its entirety.

### BACKGROUND

Hedgehog signaling is essential in many stages of development, especially in formation of left-right symmetry. Loss or reduction of hedgehog signaling leads to multiple developmental deficits and malformations, one of the most striking of which is cyclopia.

Many cancers and proliferative conditions have been shown to depend on the hedgehog pathway. The growth of such cells and survival can be affected by treatment with the compounds disclosed herein. Recently, it has been reported that activating hedgehog pathway mutations occur in sporadic basal cell carcinoma (Xie et al. (1998) Nature 391: 90-2) and primitive neuroectodermal tumors of the central nervous system (Reifenberger et al. (1998) Cancer Res 58: 1798-803). Uncontrolled activation of the hedgehog pathway has also been shown in numerous cancer types such as GI tract cancers including pancreatic, esophageal, gastric cancer (Berman et al. (2003) Nature 425: 846-51, Thayer et al. (2003) Nature 425: 851-56) lung cancer (Watkins et al. (2003) Nature 422: 313-317, prostate cancer (Karhadkar et al (2004) Nature 431: 707-12, Sheng et al. (2004) Molecular Cancer 3: 29-42, Fan et al. (2004) Endocrinology 145: 3961-70), breast cancer (Kubo et al. (2004) Cancer Research 64: 6071-74, Lewis et al. (2004) Journal of Mammary Gland Biology and Neoplasia 2: 165-181) and hepatocellular cancer (Sicklick et al. (2005) ASCO conference, Mohini et al. (2005) AACR conference).

For example, small molecule inhibition of the hedgehog pathway has been shown to inhibit the growth of basal cell carcinoma (Williams, et al., 2003 PNAS 100: 4616-21), medulloblastoma (Berman et al., 2002 Science 297: 1559-61), pancreatic cancer (Berman et al., 2003 Nature 425: 846-51), gastrointestinal cancers (Berman et al., 2003 Nature 425: 846-51, published PCT application WO 05/013800), esophageal cancer (Berman et al., 2003 Nature 425: 846-51), lung cancer (Watkins et al., 2003. Nature 422: 313-7), and prostate cancer (Karhadkar et al., 2004. Nature 431: 707-12).

In addition, it has been shown that many cancer types have uncontrolled activation of the hedgehog pathway, for example, breast cancer (Kubo et al., 2004. Cancer Research 64: 6071-4), hepatocellular cancer (Patil et al., 2005. 96th Annual AACR conference, abstract #2942 Sicklick et al., 2005. ASCO annual meeting, abstract #9610), hematological malignancies (Watkins and Matsui, unpublished results), basal cell carcinoma (Bale & Yu, 2001. Human Molec. Genet. 10:757-762 Xie et al., 1998 Nature 391: 90-92), medulloblastoma (Pietsch et al., 1997. Cancer Res. 57: 2085-88), prostate cancer (Karhadkar et al., 2003, Nature, 431:846-851), and gastric cancer (Ma et al., 2005 Carcinogenesis May 19, 2005 (Epub)).

### SUMMARY

The invention relates generally to methods of extending relapse free survival in a cancer patient who is undergoing or has undergone cancer therapy (for example, treatment with a chemotherapeutic, radiation therapy and/or surgery) by administering a therapeutically effective amount of a hedgehog signaling pathway inhibitor (hereinafter “hedgehog inhibitor”) to the patient. In some embodiments, the hedgehog inhibitor is administered concurrently with the cancer therapy. In instances of concurrent administration, the hedgehog inhibitor may continue to be administered after the cancer therapy has ceased. In other embodiments, the hedgehog inhibitor is administered after cancer therapy has ceased (i.e., with no period of overlap with the cancer treatment).

In another embodiment, the invention relates to a method of extending relapse free survival in a cancer patient who had previously undergone cancer therapy (for example, treatment with a chemotherapeutic, radiation therapy and/or surgery) by administering a therapeutically effective amount of a hedgehog inhibitor to the patient after the cancer therapy has ceased.

The cancer treated by the methods described herein can be selected from, for example, lung cancer (e.g., small cell lung cancer or non-small cell lung cancer), bladder cancer, ovarian cancer, colon cancer, acute myelogenous leukemia, and chronic myelogenous leukemia. For treatment of small cell lung cancer according to the invention, the chemotherapeutic can be selected from etoposide, carboplatin, cisplatin, irinotecan, topotecan, gemcitabine, radiation therapy, and combinations thereof. An example of suitable chemotherapeutics for treatment of non-small cell lung cancer according to the invention include vinorelbine; cisplatin; docetaxel; pemetrexed; etoposide; gemcitabine; carboplatin; targeted therapies including bevacizumab, gefitinib, erlotinib, and cetuximab; radiation therapy; and combinations thereof. For treatment of bladder cancer according to the invention, suitable chemotherapeutics include gemcitabine, cisplatin, methotrexate, vinblastin, doxorubicin, paclitaxel, docetaxel, pemetrexed, mitomycin C, 5-fluorouracil, radiation therapy, and combinations thereof. Examples of suitable chemotherapeutics for the treatment of ovarian cancer according to the invention include paclitaxel; docetaxel; carboplatin; gemcitabine; doxorubicin; topotecan; cisplatin; irinotecan; targeted therapies such as bevacizumab; radiation therapy; and combinations thereof. For treatment of colon cancer according to the invention, examples of suitable chemotherapeutics include paclitaxel; 5-fluorouracil; leucovorin; irinotecan; oxaliplatin; capecitabine; targeted therapies including bevacizumab, cetuximab, and panitumumab; radiation therapy; and combinations thereof.

In another aspect, the invention relates to a method of treating cancer in a patient wherein the patient is undergoing other cancer therapy, the method comprising detecting elevated hedgehog ligand in the patient and administering a pharmaceutically effective amount of a hedgehog antagonist to the patient. The elevated hedgehog ligand can be detected in blood, urine, circulating tumor cells, a tumor biopsy or a bone marrow biopsy. The elevated hedgehog ligand may also be detected by systemic administration of a labeled form of an antibody to a hedgehog ligand followed by imaging. The step of detecting elevated hedgehog ligand may include the steps of measuring hedgehog ligand in the patient prior to administration of the other cancer therapy, measuring hedgehog ligand in the patient after administration of the other cancer therapy, and determining if the amount of hedgehog ligand after administration of the other chemotherapy is greater than the amount of hedgehog ligand before administration of the other chemotherapy. The other cancer therapy may be, for example, a chemotherapeutic or radiation therapy.

In another aspect, the invention relates to a method of treating cancer in a patient by identifying one or more chemotherapeutics that elevate hedgehog ligand expression in a tumor, and administering a therapeutically effective amount of the one or more chemotherapeutics that elevate hedgehog ligand expression in the tumor and a therapeutically effective amount of a hedgehog inhibitor. The step of identifying the chemotherapeutics that elevate hedgehog expression can include the steps of exposing cells from the tumor to one or more chemotherapeutics in vitro and measuring hedgehog ligand in the cells.

An example of a hedgehog inhibitor is a compound of formula I:

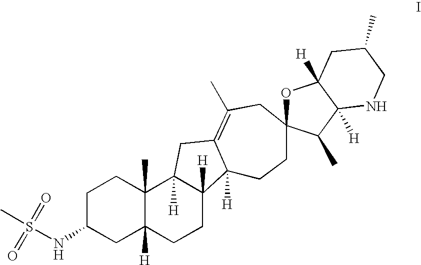

or a pharmaceutically acceptable salt thereof. An example of a pharmaceutically acceptable salt of the compound of formula I is the hydrochloride salt.

In some embodiments, the hedgehog inhibitor is administered as a pharmaceutical composition comprising the hedgehog inhibitor, or a pharmaceutically acceptable salt thereof, and a pharmaceutically acceptable excipient.

In another embodiment, the invention relates to a method of treating pancreatic cancer, by administering to a patient in need thereof a therapeutically effective amount of a compound of formula I:

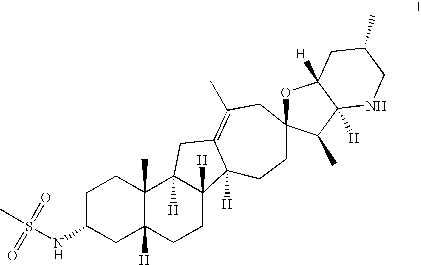

or a pharmaceutically acceptable salt thereof. An example of a therapeutically acceptable salt of the compound of formula I is a hydrochloride salt. The method can also include administration of the compound of formula I, or a pharmaceutically acceptable salt thereof, in combination with one or more chemotherapeutics (e.g., gemcitabine, cisplatin, epirubicin, 5-fluorouracil, and combinations thereof). Administration of the compound of formula I can continue after treatment with the chemotherapeutic has ceased. The compound of formula I can administered as a pharmaceutical composition comprising the compound of formula I, or a pharmaceutically acceptable salt thereof, and a pharmaceutically acceptable excipient.

### DESCRIPTION OF FIGURES

FIG. 1 is a graph depicting the change in tumor volume over time for BxPC-3 pancreatic tumor xenografts treated with vehicle and Compound 42.

FIG. 2A is a graph depicting human Gli-1 levels in BxPC-3 pancreatic tumor xenografts treated with vehicle and Compound 42.

FIG. 2A is a graph depicting murine Gli-1 levels in BxPC-3 pancreatic tumor xenografts treated with vehicle and Compound 42.

FIG. 3 is a graph depicting the change in tumor volume over time for BxPC-3 pancreatic tumor xenografts treated with vehicle, Compound 42, gemcitabine, and a combination of Compound 42 and gemcitabine.

FIG. 4 is a graph depicting the change in tumor volume over time for MiaPaCa pancreatic tumor xenografts treated with vehicle, Compound 42, gemcitabine, and a combination of Compound 42 and gemcitabine.

FIG. 5 is a graph depicting the change in tumor volume over time for LX22 small cell lung cancer tumor xenografts treated with vehicle, Compound 42, etoposide/carboplatin, and a combination of Compound 42 and etoposide/carboplatin.

FIG. 6 is a graph depicting the change in tumor volume over time for LX22 small cell lung cancer tumor xenografts treated with vehicle, Compound 42, etoposide/carboplatin followed by vehicle, and etoposide/carboplatin followed by Compound 42.

FIG. 7A is a graph depicting murine Indian hedgehog levels in LX22 small cell lung cancer tumor xenografts that were treated with etoposide/carboplatin followed by vehicle or Compound 42.

FIG. 7B is a graph depicting human Indian hedgehog levels in LX22 small cell lung cancer tumor xenografts that were treated with etoposide/carboplatin followed by vehicle or Compound 42.

FIG. 8A is a graph depicting murine Gli-1 expression levels in LX22 small cell lung cancer tumor xenografts that were treated with etoposide/carboplatin followed by vehicle or Compound 42.

FIG. 8B is a graph depicting human Gli-1 expression levels in LX22 small cell lung cancer tumor xenografts that were treated with etoposide/carboplatin followed by vehicle or Compound 42.

FIG. 9A is a graph depicting the change in murine hedgehog ligand expression levels in UMUC-3 bladder cancer tumor xenografts treated with gemcitabine as compared to naive UMUC-3 bladder cancer tumor xenografts.

FIG. 9B is a graph depicting the change in human hedgehog ligand expression levels in UMUC-3 bladder cancer tumor xenografts treated with gemcitabine as compared to naive UMUC-3 bladder cancer tumor xenografts.

FIG. 10 is a graph depicting the change in human Sonic, Indian and Desert Hedgehog ligand expression in UMUC-3 bladder cancer tumor cells treated with doxorubicin as compared to naive UMUC-3 bladder cancer tumor cells.

FIG. 11 is a graph depicting the change in human Sonic and Indian Hedgehog ligand expression in A2780 ovarian cancer tumor cells treated with carboplatin or docetaxel as compared to naive A2780 ovarian cancer tumor cells.

FIG. 12 is a graph depicting the change in human Sonic and Indian Hedgehog ligand expression in IGROV-1 ovarian cancer tumor cells treated with carboplatin or docetaxel as compared to naive IGROV-1 ovarian cancer tumor cells.

FIG. 13 is a graph depicting the change in human Sonic and Indian Hedgehog ligand expression in H82 small cell lung cancer tumor cells treated with carboplatin or docetaxel as compared to naive H82 small cell lung cancer tumor cells.

FIG. 14 is a graph depicting the change in Sonic Hedgehog ligand expression in UMUC-3 bladder cancer tumor cells exposed to hypoxic conditions as compared to UMUC-3 bladder cancer tumor cells exposed to normoxic conditions.

### DETAILED DESCRIPTION

The invention relates to methods for treating various cancers by administering hedgehog inhibitors. The hedgehog inhibitor is administered in combination with another cancer therapy, such as one or more chemotherapeutics, radiation therapy and/or surgery. The cancer therapy and hedgehog inhibitor can be administered concurrently, sequentially, or a combination of concurrent administration followed by monotherapy with the hedgehog inhibitor.

In one aspect, the invention relates to a method of treating cancer by administering to a patient a first therapeutic agent and a second therapeutic agent, wherein the second therapeutic agent is a hedgehog inhibitor. The two agents can be administered concurrently (i.e., essentially at the same time, or within the same treatment) or sequentially (i.e., one immediately following the other, or alternatively, with a gap in between administration of the two). In some embodiments, the hedgehog inhibitor is administered sequentially (i.e., after the first therapeutic). The first therapeutic agent can be a chemotherapeutic agent, or multiple chemotherapeutic agents administered sequentially or in combination. Examples of conditions that can be treated include lung cancer (e.g., small cell lung cancer or non-small cell lung cancer), bladder cancer, ovarian cancer, breast cancer, colon cancer, multiple myeloma, acute myelogenous leukemia (AML), and chronic myelogenous leukemia (CML).

In another aspect, the invention relates to a method of treating cancer including the steps of administering to a patient a first therapeutic agent, then administering the first therapeutic agent in combination with a second therapeutic agent, wherein the second therapeutic agent is a hedgehog inhibitor. Examples of conditions that can be treated include lung cancer (e.g., small cell lung cancer or non-small cell lung cancer), bladder cancer, ovarian cancer, breast cancer, colon cancer, multiple myeloma, AML and CML.

In another aspect, the invention relates to a method of treating a condition mediated by the hedgehog pathway by administering to a patient a first therapeutic agent and a second therapeutic agent, wherein the second therapeutic agent is a hedgehog inhibitor. The two agents can be administered concurrently (i.e., essentially at the same time, or within the same treatment) or sequentially (i.e., one immediately following the other, or alternatively, with a gap in between administration of the two). In some embodiments, the hedgehog inhibitor is administered sequentially (i.e., after the first therapeutic). The first therapeutic agent can be a chemotherapeutic agent. Examples of conditions that can be treated include lung cancer (e.g., small cell lung cancer or non-small cell lung cancer), bladder cancer, ovarian cancer, breast cancer, colon cancer, multiple myeloma, AML and CML.

In another aspect, the invention relates to a method of treating a condition mediated by the hedgehog pathway including the steps of administering to a patient a first therapeutic agent, then administering the first therapeutic agent in combination with a second therapeutic agent, wherein the second therapeutic agent is a hedgehog inhibitor. Examples of conditions that can be treated include lung cancer (e.g., small cell lung cancer or non-small cell lung cancer), bladder cancer, ovarian cancer, breast cancer, colon cancer, multiple myeloma, AML and CML.

The invention also relates to methods of extending relapse free survival in a cancer patient who is undergoing or has undergone cancer therapy (for example, treatment with a chemotherapeutic (including small molecules and biotherapeutics, e.g., antibodies), radiation therapy, surgery, RNAi therapy and/or antisense therapy) by administering a therapeutically effective amount of a hedgehog inhibitor to the patient. “Relapse free survival”, as understood by those skilled in the art, is the length of time following a specific point of cancer treatment during which there is no clinically-defined relapse in the cancer. In some embodiments, the hedgehog inhibitor is administered concurrently with the cancer therapy. In instances of concurrent administration, the hedgehog inhibitor may continue to be administered after the cancer therapy has ceased. In other embodiments, the hedgehog inhibitor is administered after cancer therapy has ceased (i.e., with no period of overlap with the cancer treatment). The hedgehog inhibitor may be administered immediately after cancer therapy has ceased, or there may be a gap in time (e.g., up to about a day, a week, a month, six months, or a year) between the end of cancer therapy and the administration of the hedgehog inhibitor. Treatment with the hedgehog inhibitor can continue for as long as relapse-free survival is maintained (e.g., up to about a day, a week, a month, six months, a year, two years, three years, four years, five years, or longer).

In one aspect, the invention relates to a method of extending relapse free survival in a cancer patient who had previously undergone cancer therapy (for example, treatment with a chemotherapeutic (including small molecules and biotherapeutics, e.g., antibodies), radiation therapy, surgery, RNAi therapy and/or antisense therapy) by administering a therapeutically effective amount of a hedgehog inhibitor to the patient after the cancer therapy has ceased. The hedgehog inhibitor may be administered immediately after cancer therapy has ceased, or there may be a gap in time (e.g., up to about a day, a week, a month, six months, or a year) between the end of cancer therapy and the administration of the hedgehog inhibitor.

Cancer therapies that can be combined with hedgehog inhibitors according to the invention include surgical treatments, radiation therapy, biotherapeutics (such as interferons, cytokines—e.g. Interferon α, Interferon γ, and tumor necrosis factor—hematopoietic growth factors, monoclonal serotherapy, vaccines and immunostimulants), antibodies (e.g. Avastin, Erbitux, Rituxan, and Bexxar), endocrine therapy (including peptide hormones, corticosteroids, estrogens, androgens and aromatase inhibitors), anti-estrogens (e.g. Tamoxifen, Raloxifene, and Megestrol), LHRH agonists (e.g. goscrclin and Leuprolide acetate), anti-androgens (e.g. flutamide and Bicalutamide), gene therapy, bone marrow transplantation, photodynamic therapies (e.g. vertoporfin (BPD-MA), Phthalocyanine, photosensitizer Pc4, and Demethoxy-hypocrellin A (2BA-2-DMHA)), and chemotherapeutics.

Examples of chemotherapeutics include gemcitabine, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, prednisolone, dexamethasone, cytarbine, campathecins, bleomycin, doxorubicin, idarubicin, daunorubicin, dactinomycin, plicamycin, mitoxantrone, asparaginase, vinblastine, vincristine, vinorelbine. Additional agents include nitrogen mustards (e.g. cyclophosphamide, Ifosfamide, Trofosfamide, Chlorambucil, Estramustine, and Melphalan), nitrosoureas (e.g. carmustine (BCNU) and Lomustine (CCNU)), alkylsulphonates (e.g. busulfan and Treosulfan), triazenes (e.g. Dacarbazine and Temozolomide), platinum containing compounds (e.g. Cisplatin, Carboplatin, and oxaliplatin), vinca alkaloids (e.g. vincristine, Vinblastine, Vindesine, and Vinorelbine), taxoids (e.g. paclitaxel and Docetaxol), epipodophyllins (e.g. etoposide, Teniposide, Topotecan, 9-Aminocamptothecin, Camptoirinotecan, Crisnatol, Mytomycin C, and Mytomycin C), anti-metabolites, DHFR inhibitors (e.g. methotrexate and Trimetrexate), IMP dehydrogenase Inhibitors (e.g. mycophenolic acid, Tiazofurin, Ribavirin, and EICAR), ribonucleotide reductase Inhibitors (e.g. hydroxyurea and Deferoxamine), uracil analogs (e.g. Fluorouracil, Floxuridine, Doxifluridine, Ratitrexed, and Capecitabine), cytosine analogs (e.g. cytarabine (ara C), Cytosine arabinoside, and Fludarabine), purine analogs (e.g. mercaptopurine and Thioguanine), Vitamin D3 analogs (e.g. EB 1089, CB 1093, and KH 1060), isoprenylation inhibitors (e.g. Lovastatin), dopaminergic neurotoxins (e.g. 1-methyl-4-phenylpyridinium ion), cell cycle inhibitors (e.g. staurosporine), actinomycins (e.g. Actinomycin D and Dactinomycin), bleomycins (e.g. bleomycin A2, Bleomycin B2, and Peplomycin), anthracyclines (e.g. daunorubicin, Doxorubicin (adriamycin), Idarubicin, Epirubicin, Pirarubicin, Zorubicin, and Mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., erlotinib, gefitinib, sorafenib, sunitinib), and proteasome inhibitors such as bortezomib.

Proliferative disorders and cancers that can be treated using the methods disclosed herein include, for example, lung cancer (including small cell lung cancer and non small cell lung cancer), other cancers of the pulmonary system, medulloblastoma and other brain cancers, pancreatic cancer, basal cell carcinoma, breast cancer, prostate cancer and other genitourinary cancers, gastrointestinal stromal tumor (GIST) and other cancers of the gastrointestinal tract, colon cancer, colorectal cancer, ovarian cancer, cancers of the hematopoietic system (including multiple myeloma, acute lymphocytic leukemia, acute myelocytic leukemia, chronic myelocytic leukemia, chronic lymphocytic leukemia, Hodgkin lymphoma, non-Hodgkin lymphoma, and myelodysplastic syndrome), polycythemia Vera, Waldenstrom's macroglobulinemia, heavy chain disease, soft-tissue sarcomas, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, melanoma, and other skin cancers, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, bladder carcinoma, and other genitourinary cancers, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, endometrial cancer, follicular lymphoma, diffuse large B-cell lymphoma, mantle cell lymphoma, hepatocellular carcinoma, thyroid cancer, gastric cancer, esophageal cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, thyroid cancer, neuroendocrine cancers, and carcinoid tumors.

Certain methods of the current invention may be especially effective in treating cancers that respond well to existing chemotherapies, but suffer from a high relapse rate. In these instances, treatment with the hedgehog inhibitor can increase the relapse-free survival time or rate of the patient. Examples of such cancers include lung cancer (e.g., small cell lung cancer or non-small cell lung cancer), bladder cancer, ovarian cancer, breast cancer, colon cancer, multiple myeloma, acute myelogenous leukemia (AML) and chronic myelogenous leukemia (CML).

The invention also encompasses the use of a chemotherapeutic agent and a hedgehog inhibitor for preparation of one or more medicaments for use in a method of extending relapse free survival in a cancer patient. The invention also relates to the use of a hedgehog inhibitor in the preparation of a medicament for use in a method of extending relapse free survival in a cancer patient who had previously been treated with a chemotherapeutic. The invention also encompasses the use of a hedgehog inhibitor in the preparation of a medicament for use in a method of treating pancreatic cancer patient.

It has been discovered that multiple tumor types exhibit up-regulation of Hh ligands post chemotherapy (see Examples 11 and 12 herein) and in response to other stress, such as hypoxia (see Example 12). The type of Hh ligand that is up-regulated (i.e., Sonic, Indian and/or Desert) and the degree of up-regulation vary depending upon the tumor type and the chemotherapeutic agent. Without wishing to be bound to any theory, these results suggest that stress (including chemotherapy) induces Hedgehog ligand production in tumor cells as a protective or survival mechanism. The results further suggest that up-regulation of tumor-derived Hh ligand post-chemotherapy may confer upon the surviving cell population a dependency upon the Hh pathway that is important for tumor recurrence, and thus may be susceptible to Hh pathway inhibition.

Thus, an aspect of the invention is a method of treating cancer by determining whether expression of one or more hedgehog ligands has increased during or after chemotherapy, then administering a hedgehog inhibitor. Ligand expression can be measured by detection of a soluble form of the ligand in peripheral blood and/or urine (e.g., by an ELISA assay or radioimmunoassay), in circulating tumor cells (e.g., by a fluorescence-activated cell sorting (FACS) assay, an immunohistochemisty assay, or a reverse transcription polymerase chain reaction (RT-PCR) assay), or in tumor or bone marrow biopsies (e.g., by an immunohistochemistry assay, a RT-PCR assay, or by in situ hybridization). Detection of hedgehog ligand in a given patient tumor could also be assessed in vivo, by systemic administration of a labeled form of an antibody to a hedgehog ligand followed by imaging, similar to detection of PSMA in prostate cancer patients (Bander, N.H. Nat Clin Pract Urol 2006; 3:216-225). Expression levels in a patient can be measured at least at two time-points to determine of ligand induction has occurred. For example, hedgehog ligand expression may be measured pre- and post-chemotherapy, pre-chemotherapy and at one or more time-points while chemotherapy is ongoing, or at two or more different time-points while chemotherapy is ongoing. If a hedgehog ligand is found to be up-regulated, a hedgehog inhibitor can be administered. Thus, measurement of hedgehog ligand induction in the patient can determine whether the patient receives a hedgehog pathway inhibitor in combination with or following other chemotherapy.

Another aspect of the invention relates to a method of treating cancer in a patient by identifying one or more chemotherapeutics that elevate hedgehog ligand expression in the cancer tumor, and administering one or more of the chemotherapeutics that elevate hedgehog ligand expression and a hedgehog inhibitor. To determine which chemotherapeutics elevate hedgehog expression, tumor cells can be removed from a patient prior to therapy and exposed to a panel of chemotherapeutics ex vivo and assayed to measure changes in hedgehog ligand expression (see, e.g., Am. J. Obstet. Gynecol. November 2003, 189(5):1301-7; J. Neurooncol., February 2004, 66(3):365-75). A chemotherapeutic that causes an increase in one or more hedgehog ligands is then administered to the patient. A chemotherapeutic that causes an increase in one or more hedgehog ligands may be administered alone or in combination with one or more different chemotherapeutics that may or may not cause an increase in one or more hedgehog ligands. The hedgehog inhibitor and chemotherapeutic can be administered concurrently (i.e., essentially at the same time, or within the same treatment) or sequentially (i.e., one immediately following the other, or alternatively, with a gap in between administration of the two). Treatment with the hedgehog inhibitor may continue after treatment with the chemotherapeutic ceases. Thus, the chemotherapeutic is chosen based upon its ability to up-regulate hedgehog ligand expression (which, in turn, renders the tumors dependent upon the hedgehog pathway), which may make the tumor susceptible to treatment with a hedgehog inhibitor.

Suitable hedgehog inhibitors include, for example, those described and disclosed in U.S. Pat. No. 7,230,004, U.S. Patent Application Publication No. 2008/0293754, U.S. Patent Application Publication No. 2008/0287420, and U.S. Patent Application Publication No. 2008/0293755, the entire disclosures of which are incorporated by reference herein. Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354.

For example, the hedgehog inhibitor can be a compound having the following structure:

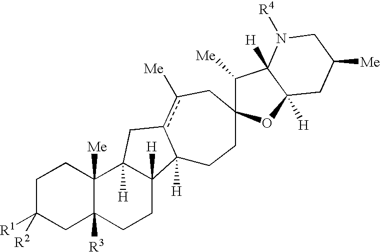

or a pharmaceutically acceptable salt thereof; wherein

R¹ is H, alkyl, —OR, amino, sulfonamido, sulfamido, —OC(O)R⁵, —N(R⁵)C(O)R⁵, or a sugar;

R² is H, alkyl, alkenyl, alkynyl, aryl, cycloalkyl, nitrile, or heterocycloalkyl;

or R¹ and R² taken together form ═O, ═S, ═N(OR), ═N(R), ═N(NR₂), or ═C(R)₂;

R³ is H, alkyl, alkenyl, or alkynyl;

R⁴ is H, alkyl, alkenyl, alkynyl, aryl, cycloalkyl, heterocycloalkyl, aralkyl, heteroaryl, heteroaralkyl, haloalkyl, —OR, —C(O)R⁵, —CO₂R⁵, —SO₂R⁵, —C(O)N(R⁵)(R⁵), —[C(R)₂]_{q}—R⁵, [(W)—N(R)C(O)]_{q}R⁵, —[(W)—C(O)]_{q}R⁵, —[(W)—C(O)O]_{q}R⁵, —[(W)—OC(O)]_{q}R⁵, —[(W)—SO₂]_{q}R⁵, —[(W)—N(R⁵)SO₂]_{q}R⁵, —[(W)—C(O)N(R⁵)]_{q}R⁵, —[(W)—O]_{q}R⁵, —[(W)—N(R)]_{q}R⁵, —W—NR₃⁺X⁻ or —[(W)—S]_{q}R⁵;

each W is independently for each occurrence a diradical;

each q is independently for each occurrence 1, 2, 3, 4, 5, or 6;

X⁻ is a halide;

each R⁵ is independently for each occurrence H, alkyl, alkenyl, alkynyl, aryl, cycloalkyl, heterocycloalkyl, aralkyl, heteroaryl, heteroaralkyl or —[C(R)₂]ₚ—R⁶;

or any two occurrences of R⁵ on the same substituent can be taken together to form a 4-8 membered optionally substituted ring which contains 0-3 heteroatoms selected from N, O, S, and P;

p is 0-6;

each R⁶ is independently hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —OP(O)(OR)(OR), —NP(O)(OR)(OR), or —P(O)(OR)(OR);

provided that when R², R³ are H and R⁴ is hydroxyl; R¹ can not be hydroxyl;

provided that when R², R³, and R⁴ are H; R¹ can not be hydroxyl; and

provided that when R², R³, and R⁴ are H; R¹ can not be sugar.

Examples of compounds include:

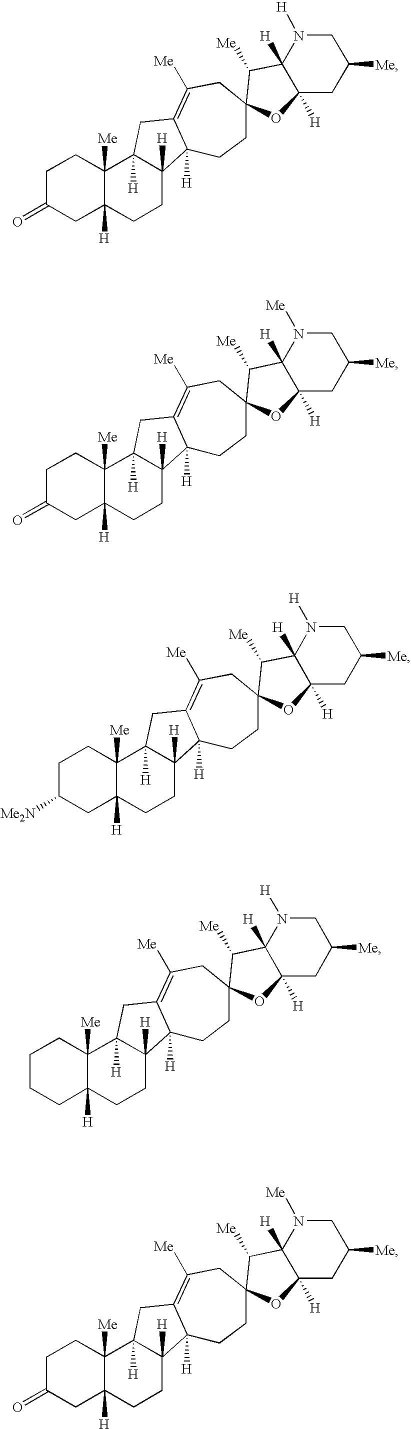

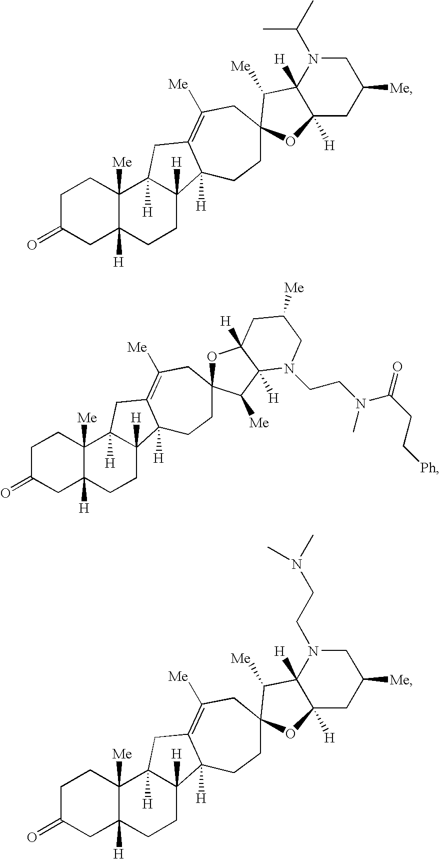

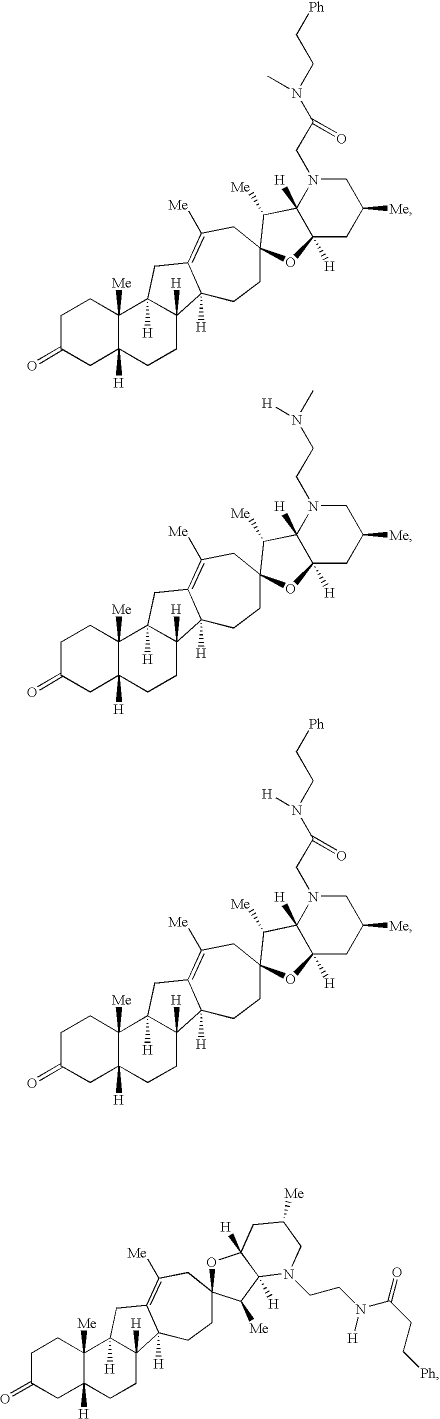

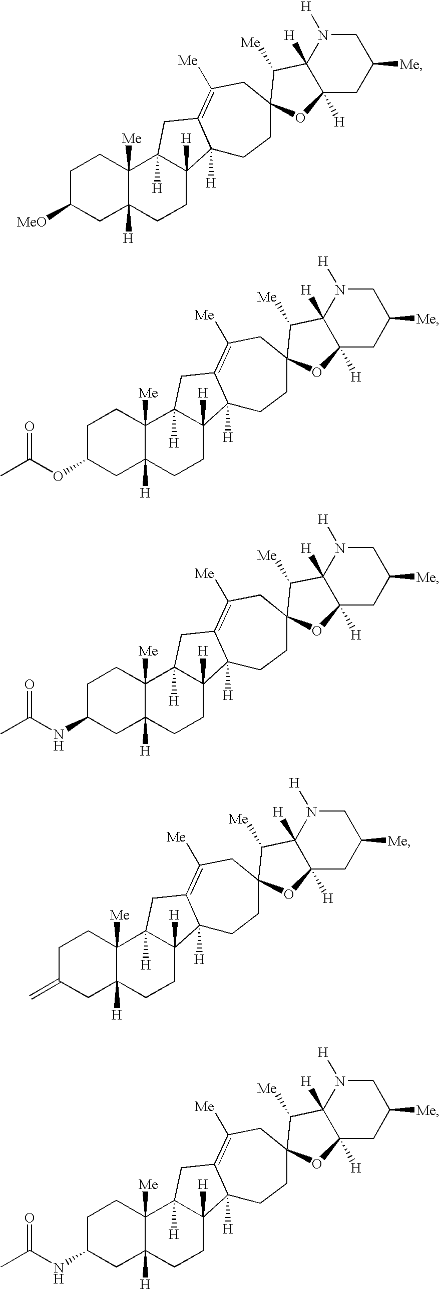

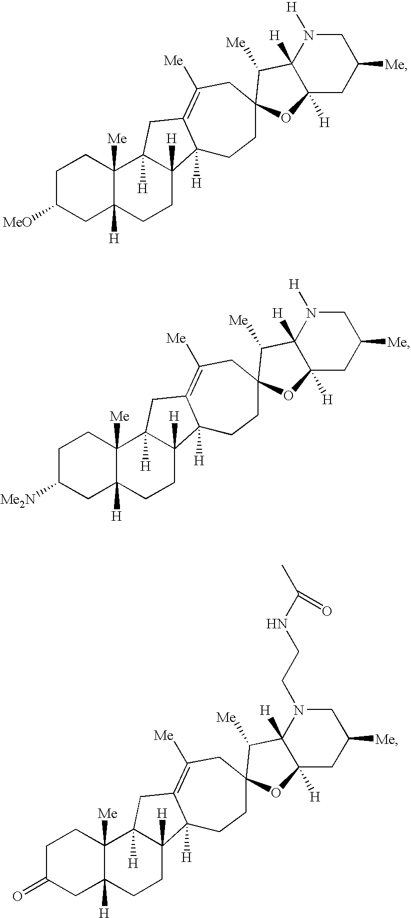

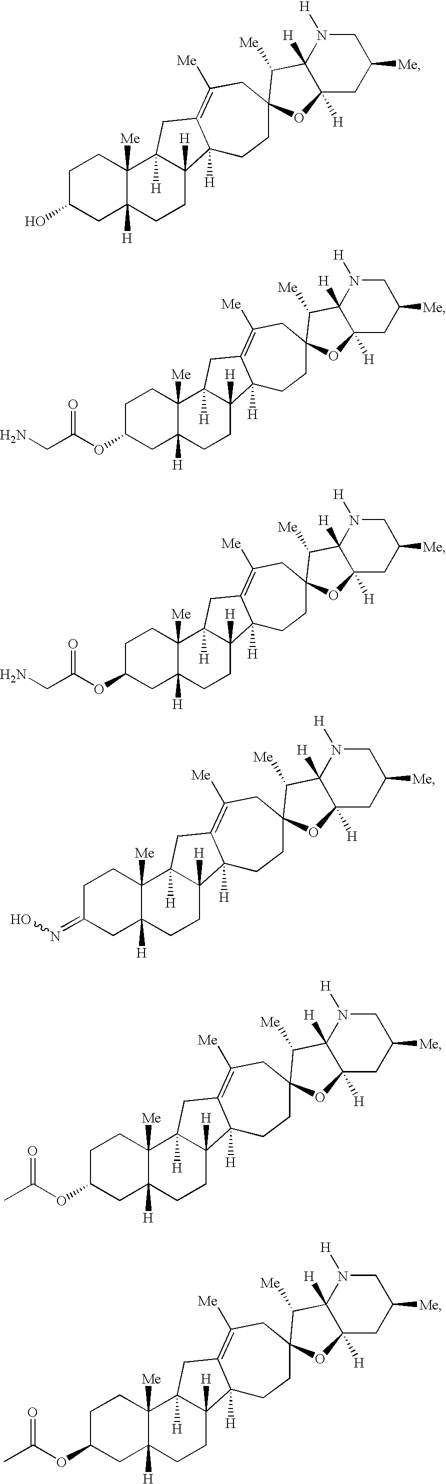

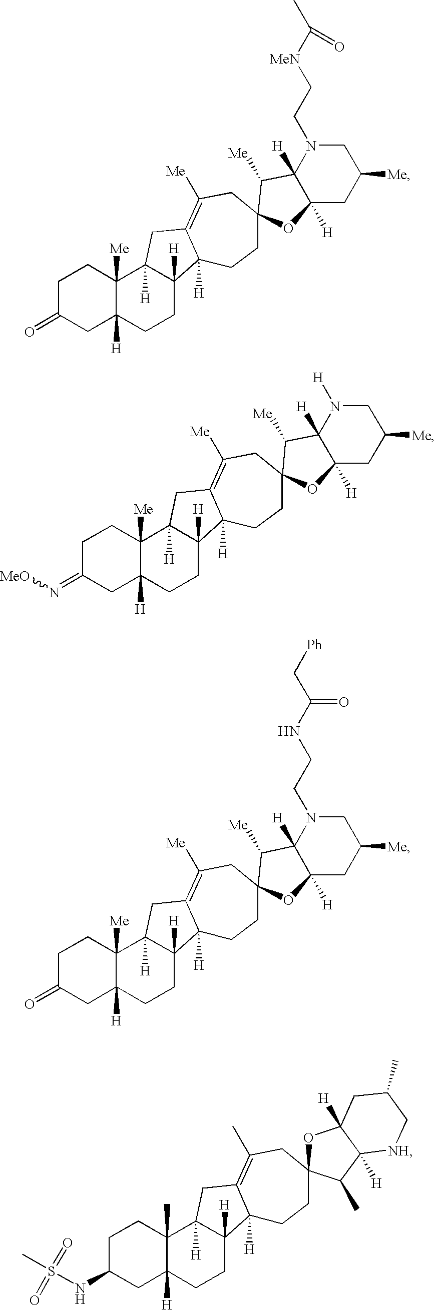

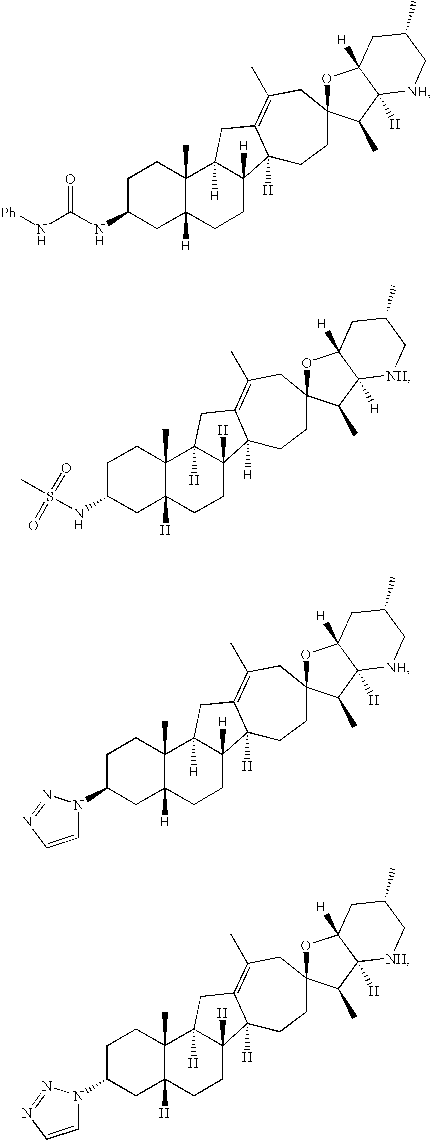

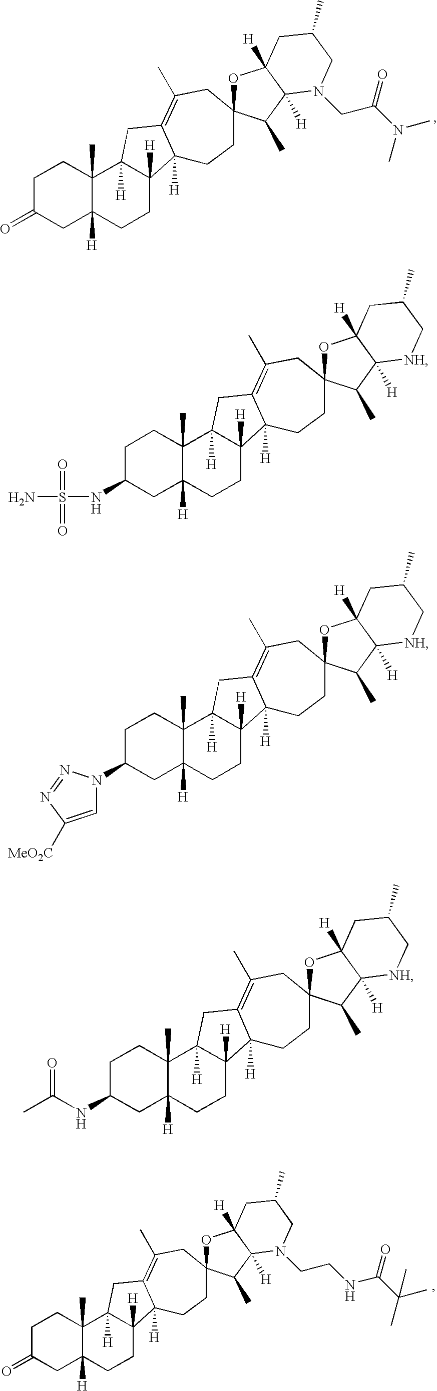

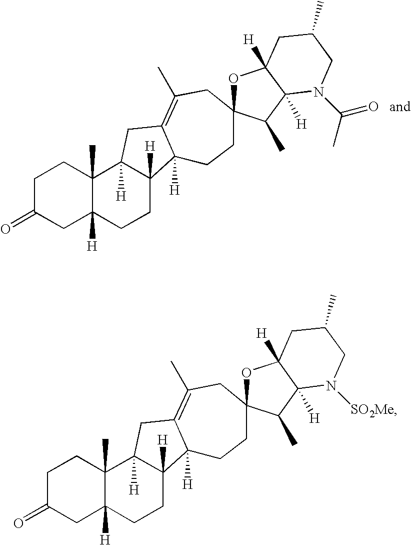

and pharmaceutically acceptable salts thereof.

One example of a suitable hedgehog inhibitor for the methods of the current invention is the compound of formula I:

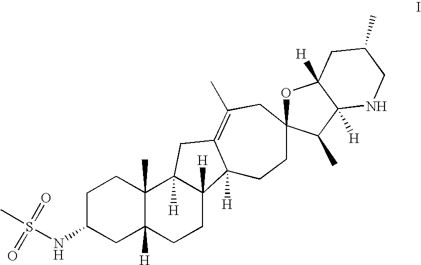

or a pharmaceutically acceptable salt thereof. An example of a pharmaceutically acceptable salt is a hydrochloride salt of the compound of formula I.

Hedgehog inhibitors useful in the current invention may contain a basic functional group, such as amino or alkylamino, and are thus capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately treating the compound in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, besylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the present invention include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, benzenesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately treating the compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

To practice the methods of the invention, the hedgehog inhibitor and/or the chemotherapeutic agent may be delivered in the form of pharmaceutically acceptable compositions which comprise a therapeutically-effective amount of one or more hedgehog inhibitors and/or one or more chemotherapeutic formulated together with one or more pharmaceutically acceptable excipients. In some instances, the hedgehog inhibitor and the chemotherapeutic agent are administered in separate pharmaceutical compositions and may (e.g., because of different physical and/or chemical characteristics) be administered by different routes (e.g., one therapeutic is administered orally, while the other is administered intravenously). In other instances, the hedgehog inhibitor and the chemotherapeutic may be administered separately, but via the same route (e.g., both orally or both intravenously). In still other instances, the hedgehog inhibitor and the chemotherapeutic may be administered in the same pharmaceutical composition.

Pharmaceutical compositions may be specially formulated for administration in solid or liquid form, including those adapted for the following: oral administration, for example, drenches (aqueous or non-aqueous solutions or suspensions), tablets (e.g., those targeted for buccal, sublingual, and systemic absorption), capsules, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin; intravaginally or intrarectally, for example, as a pessary, cream or foam; sublingually; ocularly; transdermally; pulmonarily; or nasally.

Examples of suitable aqueous and nonaqueous carriers which may be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, dispersing agents, lubricants, and/or antioxidants. Prevention of the action of microorganisms upon the compounds of the present invention may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Methods of preparing these formulations or compositions include the step of bringing into association the hedgehog inhibitor and/or the chemotherapeutic with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

The hedgehog inhibitors and the chemotherapeutics of the present invention can be given per se or as a pharmaceutical composition containing, for example, about 0.1 to 99%, or about 10 to 50%, or about 10 to 40%, or about 10 to 30%, or about 10 to 20%, or about 10 to 15% of active ingredient in combination with a pharmaceutically acceptable carrier. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including, for example, the activity of the particular compound employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a hedgehog inhibitor and/or a chemotherapeutic will be that amount of the compound which is the lowest dose effective to produce a therapeutic effect. Such an effective dose will generally depend upon the factors described above. Generally, oral, intravenous and subcutaneous doses of the compounds of the present invention for a patient, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to about 500 mg per day, or about 0.1 mg to about 500 mg per day.

The subject receiving this treatment is any animal in need, including primates, in particular humans, equines, cattle, swine, sheep, poultry, dogs, cats, mice and rats.

The compounds can be administered daily, every other day, three times a week, twice a week, weekly, or bi-weekly. The dosing schedule can include a “drug holiday,” i.e., the drug can be administered for two weeks on, one week off, or three weeks on, one week off, or four weeks on, one week off, etc., or continuously, without a drug holiday. The compounds can be administered orally, intravenously, intraperitoneally, topically, transdermally, intramuscularly, subcutaneously, intranasally, sublingually, or by any other route.

Since the hedgehog inhibitors are administered in combination with other treatments (such as additional chemotherapeutics, radiation or surgery) the doses of each agent or therapy may be lower than the corresponding dose for single-agent therapy. The dose for single-agent therapy can range from, for example, about 0.0001 to about 200 mg, or about 0.001 to about 100 mg, or about 0.01 to about 100 mg, or about 0.1 to about 100 mg, or about 1 to about 50 mg per kilogram of body weight per day. The determination of the mode of administration and the correct dosage is well within the knowledge of the skilled clinician.

The invention now being generally described, it will be more readily understood by reference to the following examples, which are included merely for purposes of illustration of certain aspects and embodiments of the present invention, and are not intended to limit the invention.

### Example 1

### Activity in the Hedgehog Pathway

Hedgehog pathway specific cancer cell killing effects may be ascertained using the following assay. C3H10T1/2 cells differentiate into osteoblasts when contacted with the sonic hedgehog peptide (Shh-N). Upon differentiation, these osteoblasts produce high levels of alkaline phosphatase (AP) which can be measured in an enzymatic assay (Nakamura et al., 1997 BBRC 237: 465). Compounds that block the differentiation of C3H10T1/2 into osteoblasts (a Shh dependent event) can therefore be identified by a reduction in AP production (van der Horst et al., 2003 Bone 33: 899). The assay details are described below.

### Cell Culture

Mouse embryonic mesoderm fibroblasts C3H10T1/2 cells (obtained from ATCC) were cultured in Basal MEM Media (Gibco/Invitrogen) supplemented with 10% heat inactivated FBS (Hyclone), 50 units/ml penicillin and 50 ug/ml streptomycin (Gibco/Invitrogen) at 37° C. with 5% CO2 in air atmosphere.

### Alkaline Phosphatase Assay

C3H10T1/2 cells were plated in 96 wells with a density of 8×103 cells/well. Cells were grown to confluence (72 hrs.). After sonic hedgehog (250 ng/ml) and/or compound treatment, the cells were lysed in 110 μL of lysis buffer (50 mM Tris pH 7.4, 0.1% TritonX100), plates were sonicated and lysates spun through 0.2 μm PVDF plates (Corning). 40 μL of lysates was assayed for AP activity in alkaline buffer solution (Sigma) containing 1 mg/ml p-Nitrophenyl Phosphate. After incubating for 30 min at 37° C., the plates were read on an Envision plate reader at 405 nm. Total protein was quantified with a BCA protein assay kit from Pierce according to manufacturer's instructions. AP activity was normalized against total protein. Using the above-described assay, Compound 42 was shown to be an antagonist of the hedgehog pathway with an IC50 less than 20 nM.

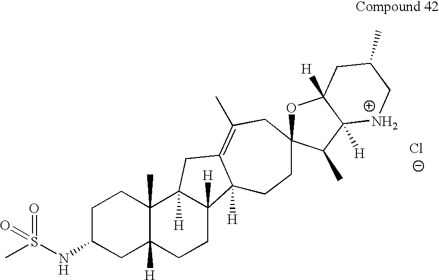

### Example 2

### Pancreatic Cancer Monotherapy Model

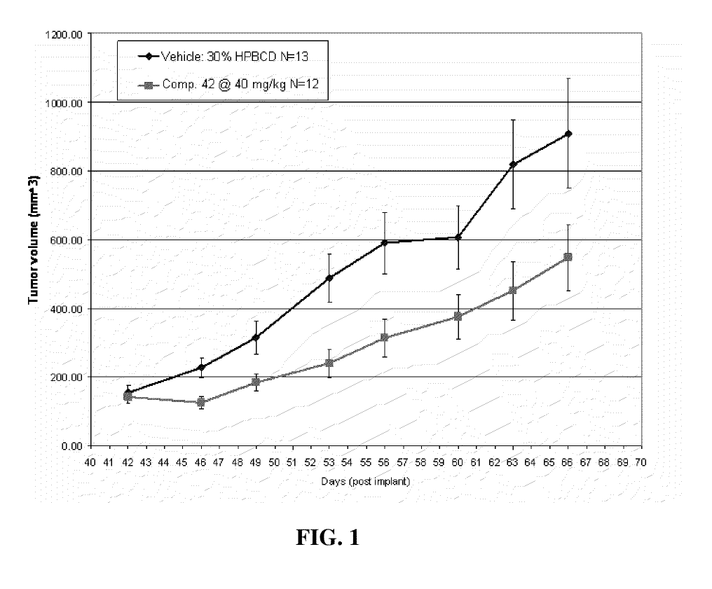

The activity of Compound 42 was tested in a human pancreatic model. BxPC-3 cells were implanted subcutaneously into the flanks of the right legs of mice. On day 42 post-tumor implant, the mice were randomized into two groups to receive either Vehicle (30% HPBCD) or Compound 42. Compound 42 was dosed at 40 mg/kg/day. After receiving 25 daily doses, Compound 42 statistically reduced tumor volume growth by about 40% when compared to the vehicle control (p=0.0309) (see FIG. 1).

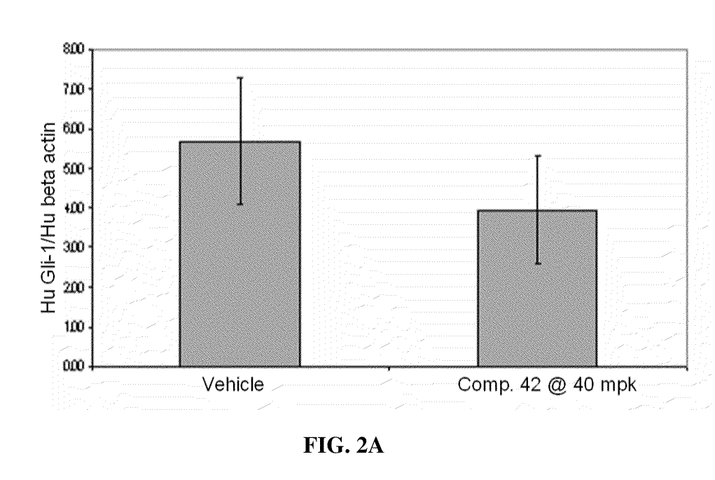

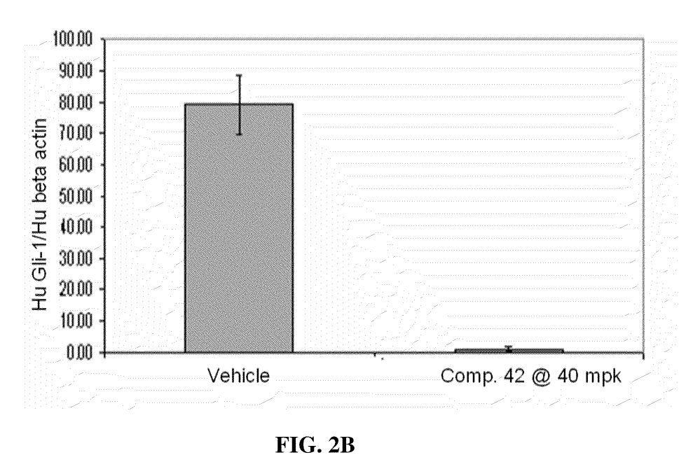

At the end of the study, the tumors were harvested 4 hours post the last dose to evaluate an on target response by q-RT-PCR analysis of the Hedgehog pathway genes. As shown in FIG. 2A, Human Gli-1 was not modulated in either the vehicle or the treated group. However, murine Gli-1 mRNA levels were significantly down-regulated in the Compound 42 treated group when compared to the vehicle treated group (see FIG. 2B).

### Example 3

### Pancreatic Cancer Concurrent Combination Therapy Model

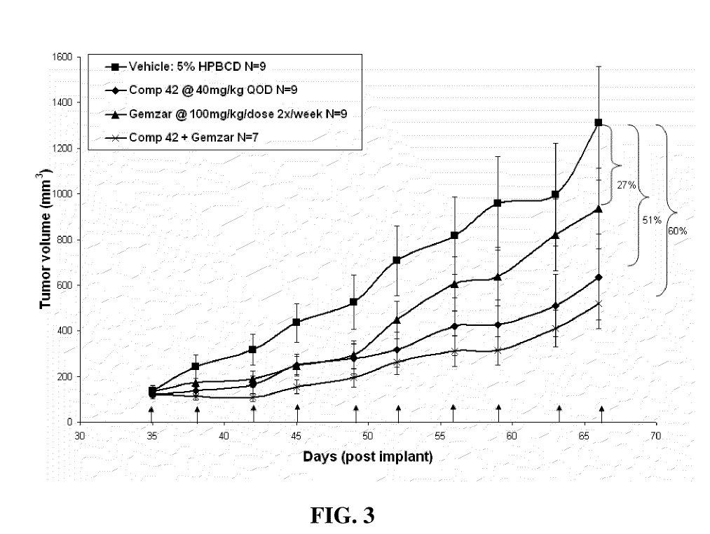

Animals bearing BxPC-3 pancreatic cancer xenografts were treated with the chemotherapeutic drug gemcitabine in concurrent combination with Compound 42. Gemcitabine was administered at a dose of 100 mg/kg twice weekly by intraperitoneal injection while Compound 42 was administered at a dose of 40 mg/kg daily by oral gavage. As shown in FIG. 3, under these conditions the tumors showed a 33% response to gemcitabine alone, a 55% response to Compound 42 alone, and a 67% response to the combination of Compound 42 and gemcitabine.

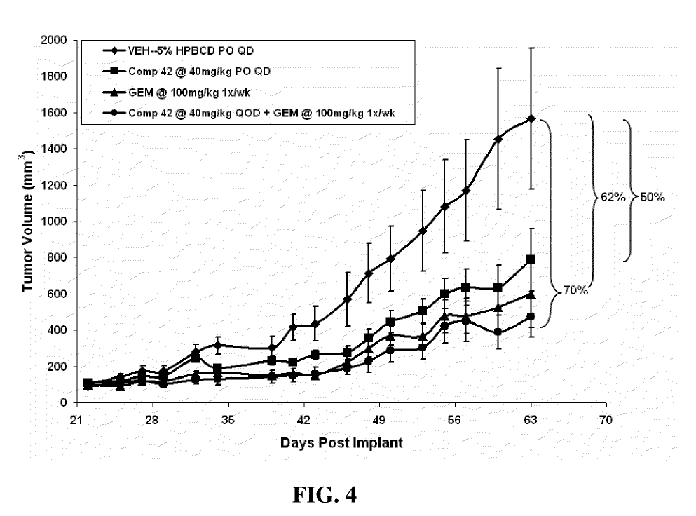

In another model, Animals bearing MiaPaCa pancreatic cancer xenografts were treated with the chemotherapeutic drug gemcitabine in concurrent combination with Compound 42. Gemcitabine was administered at a dose of 100 mg/kg once weekly by intraperitoneal injection while Compound 42 was administered at a dose of 40 mg/kg daily by oral gavage. As shown in FIG. 4, under these conditions the tumors showed a 52% response to gemcitabine alone, a 50% response to Compound 42 alone, and a 70% response to the combination of Compound 42 and gemcitabine.

### Example 4

### Lung Cancer Concurrent Combination Therapy Model

To test the activity of Compound 42 in a human small cell lung cancer tumor model, LX22 cells were implanted subcutaneously into the flank of the right leg of male Ncr nude mice. LX22 is primary xenograft model of SCLC derived from chemo-naive patients, which has been maintained by mouse to mouse passaging. This tumor responds to etoposide/carboplatin chemotherapy in way that closely resembles a clinical setting. LX22 regresses during chemotherapy treatment, goes through a period of remission, and then begins to recur.

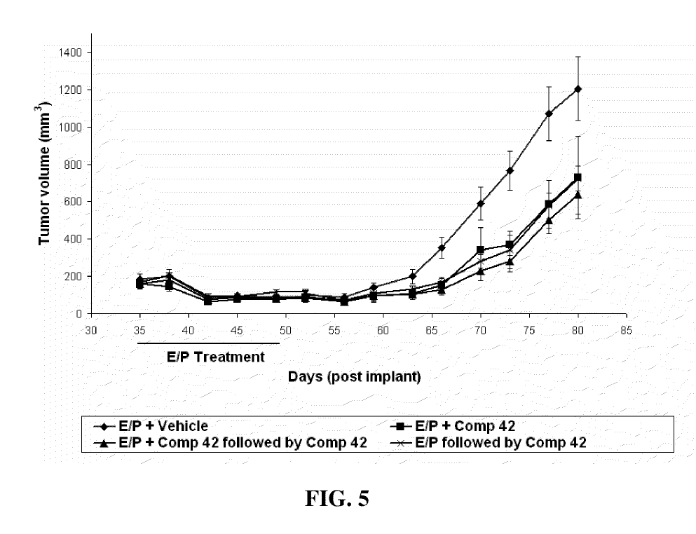

Animals bearing LX-22 small cell lung cancer xenografts were treated with the chemotherapeutic drugs etoposide and carboplatin in concurrent combination with Compound 42. In this experiment, etoposide was administered at a dose of 12 mg/kg by intravenous route on three consecutive days followed by a single administration two weeks after the initial dose. Carboplatin was administered at a dose of 60 mg/kg weekly for three weeks by intravenous injection. Compound 42 was administered at a dose of 40 mg/kg daily by oral gavage either at the same time as etoposide/carboplatin or immediately following etoposide/carboplatin treatment. As shown in FIG. 5, under these conditions the tumors showed an overall 40% response to all treatments when compared to those animals receiving etoposide/carboplatin alone.

### Example 5

### Chemo-Resistant Recurrence Model

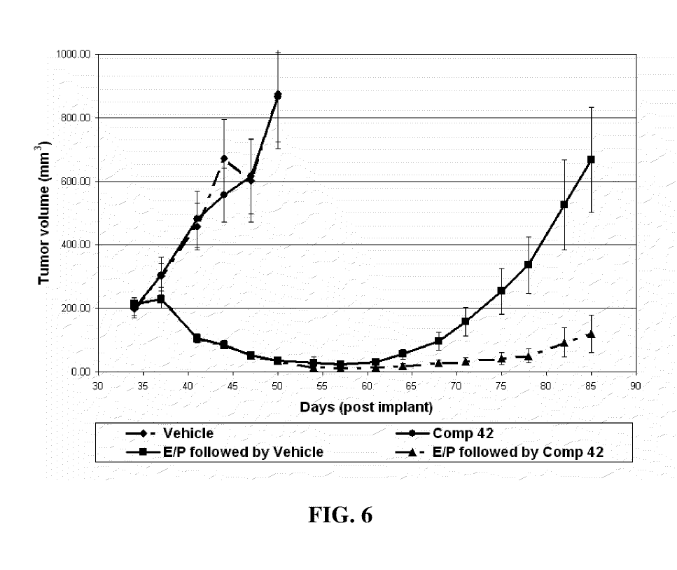

In the LX22 model, Compound 42 single agent activity and its ability to modulate the chemo-resistant recurrence were tested. On day 32 post tumor implant, mice were randomized into three dosing groups to receive vehicle (30% HBPCD), Compound 42, or the chemotherapy combination of etoposide and carboplatin (E/P). Compound 42 was administered at a dose of 40 mg/kg/day, etoposide was administered i.v. at 12 mg/kg on days 34, 35, 36, and 48, and carboplatin was administered i.v. at 60 mg/kg on days 34, 41, and 48, post tumor implant. After 16 consecutive doses there was no measurable difference between the group treated with Compound 42 and the vehicle treated group (see FIG. 6). On day 50, the E/P treated mice were further randomized to receive either vehicle (30% HPBCD) or Compound 42 follow-up treatment. Compound 42 was administered at 40 mg/kg/day. As shown in FIG. 6, after 35 consecutive doses of Compound 42, there was a substantial delay in tumor recurrence in the treated group (82%), compared to the vehicle group (p=0.0101).

### Example 6

### Colon Cancer Combination Therapy Model

Animals bearing Colo205 colon cancer xenografts were treated with the chemotherapeutic drug 5-fluorouracil in combination with Compound 42. 5-fluorouracil was administered at a dose of either 50 mg/kg or 100 mg/kg as a once weekly intraperitoneal injection for two weeks. Compound 42 was administered at 40 mg/kg as a daily oral gavage for 21 days. Under these conditions the tumors showed a 68% to 5-fluorouracil alone or in combination with Compound 42.

### Example 7

### Colon Cancer Chemo-Resistant Recurrence Models

Animals are implanted with SW620 colon cancer cells. Tumor bearing animals are administered paclitaxel for such a time that their tumors respond to chemotherapy treatment. These animals are randomized into two groups, one receiving vehicle and one receiving Compound 42. Tumor response to the different therapies is determined as discussed herein.

Alternatively, Colo205 colon cancer cells are implanted into experimental animals. Tumor bearing animals will be administered 5-fluorouracil for such a time that their tumors respond to chemotherapy treatment. These animals are then randomized into two groups, one receiving vehicle and one receiving Compound 42. Tumor response to the different therapies is determined as discussed herein.

### Example 8

### Ovarian Cancer Models

Mice bearing IGROV-1 ovarian cancer xenografts were treated with daily doses of Compound 42 at 40 mg/kg for 21 consecutive days. No substantive effect on tumor growth was observed at this dosage with this particular ovarian cancer cell xenograft. In a further study, mice bearing IGROV-1 ovarian cancer xenografts were treated with 5 consecutive daily doses of paclitaxel at 15 mg/kg followed by Compound 42 at 40 mg/kg for 21 consecutive days. Again, no substantive effect on tumor growth was observed at these dosages with this particular ovarian cancer cell xenograft.

To determine if other ovarian cancer cell types respond to treatment with Compound 42, SKOV-3, OVCAR-4 or OVCAR-5 ovarian cancer cells are implanted into experimental animals. To determine the effect of monotherapy and concurrent combination therapy, tumor bearing animals are administered paclitaxel or carboplatin alone, Compound 42 alone, or Compound 42 and paclitaxel or carboplatin in combination. To determine the effect of sequential combination therapy, tumor bearing animals are administered paclitaxel or carboplatin for such a time that their tumors respond to chemotherapy treatment. These animals are then randomized into two groups, one receiving vehicle and one receiving Compound 42. Tumor response to the different therapies is determined as discussed herein.

### Example 9

### Bladder Cancer Models

To determine the effect of monotherapy and concurrent combination therapy, animals are implanted with UMUC-3 bladder cancer cells. Tumor bearing animals are then administered gemcitabine/cisplatin alone, Compound 42 alone, or the three agents in combination. Tumor response to the different therapies is determined as discussed herein.

To determine the effect of sequential combination therapy, animals are implanted with UMUC-3 bladder cancer cells, and tumor bearing animals are then administered a combination of gemcitabine and cisplatin for such a time that their tumors respond to chemotherapy treatment. These animals are then randomized into two groups, one receiving vehicle and one receiving Compound 42. Tumor response to the different therapies is determined as discussed herein.

Alternatively, SW780 bladder cancer cells are implanted into experimental animals. To determine the effect of monotherapy and concurrent combination therapy, tumor bearing animals are administered gemcitabine/cisplatin alone, Compound 42 alone, or the three agents in combination. To determine the effect of sequential combination therapy, tumor bearing animals are administered a combination of gemcitabine and cisplatin for such a time that their tumors respond to chemotherapy treatment. These animals are then randomized into two groups, one receiving vehicle and one receiving Compound 42. Tumor response to the different therapies is determined as discussed herein.

### Example 10

### Non-Small Cell Cancer Models

To determine the effect of monotherapy and concurrent combination therapy, animals are implanted with NCI-H1650 non-small cell lung cancer cells. Tumor bearing animals are then administered gefitinib alone, Compound 42 alone, or the two agents in combination. Tumor response to the different therapies is determined as discussed herein.

To determine the effect of sequential combination therapy, animals are implanted with NCI-H1650 non-small cell lung cancer cells, and tumor bearing animals are then administered gefitinib for such a time that their tumors respond to gefitinib treatment. These animals are then randomized into two groups, one receiving vehicle and one receiving Compound 42. Tumor response to the different therapies is determined as discussed herein.

### Example 11

### Hedgehog Ligand Induction Studies

Follow up studies in the LX22 model were designed to examine Hh pathway modulation by Compound 42 post etoposide and carboplatin (E/P) treatment. As described in Example 4 above, animals bearing LX22 small cell lung cancer xenografts were treated with etoposide and carboplatin. A single dose of Compound 42 (40 mg/kg) was administered 24 hours prior to each time point collected. Naïve tumors were collected from five animals for baseline levels prior to chemotherapy treatment. Tumors from four animals were collected on days 1, 4, 7, and 10, and tumors from three animals were collected on day 14. Samples were collected for q-RT-PCR analysis and histology/immunohistochemistry evaluation. RNA was extracted and q-RT-PCR analysis was completed by first converting to cDNA then using the one-step master mix (FAST method on 7900).

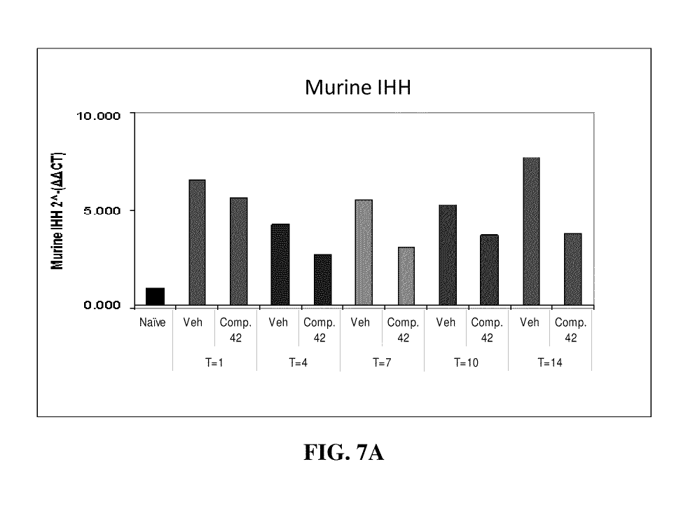

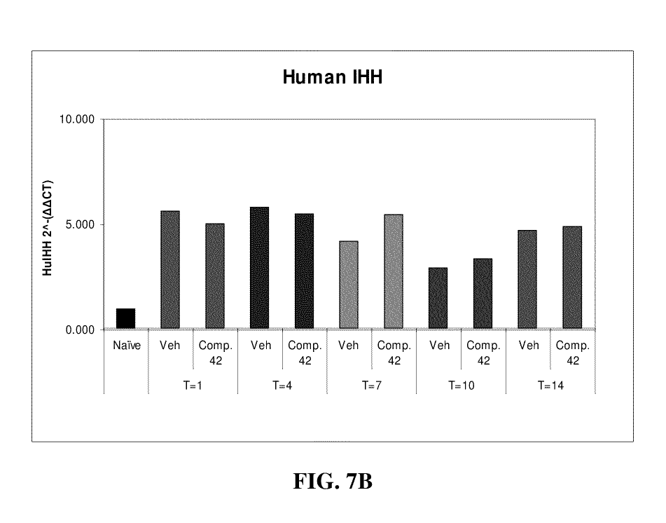

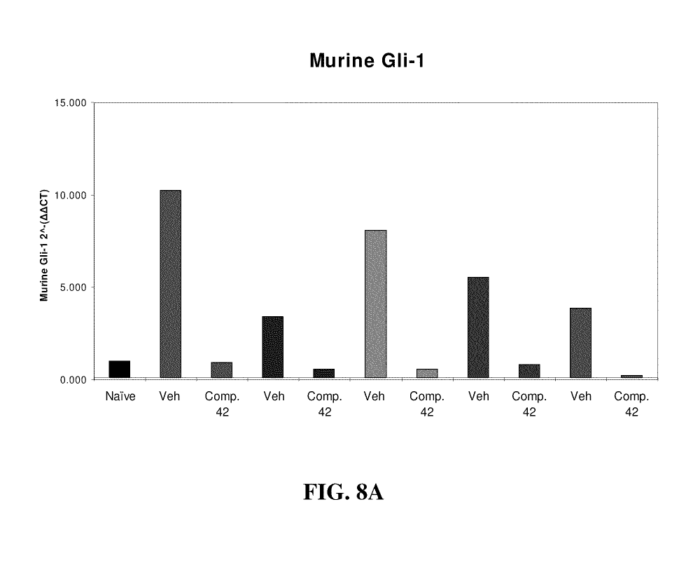

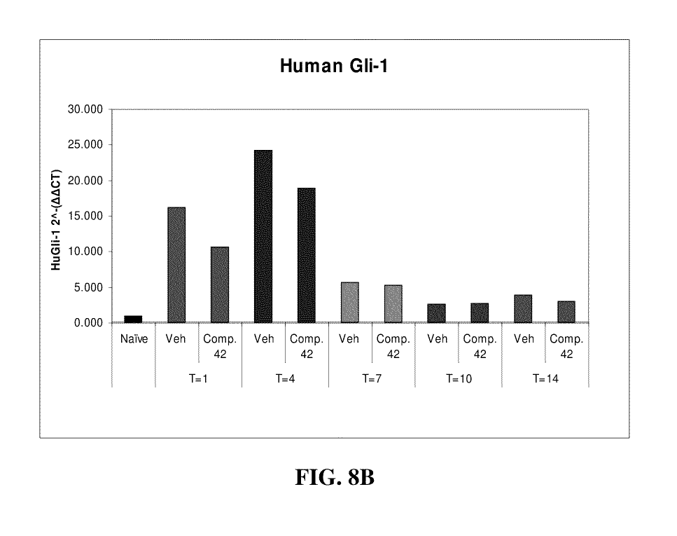

The results of this study showed that Hh ligand, specifically Indian Hh (IHH), was up-regulated in the human tumor cells and the surrounding murine stroma cells following chemotherapy, as measured both by RT-PCR and immunohistochemistry (see FIGS. 7A and 7B). In addition, stromal-derived murine Gli-1 and tumor-derived human Gli-1 were induced in response to tumor-derived ligand. Murine Gli-1 expression remained elevated compared to the expression level in naive tumors for at least 14 days post the cessation of E/P treatment and was inhibited by administration of Compound 42 (see FIG. 8A), while human Gli-1 expression was not affected by administration of Compound 42 (see FIG. 8B). Without wishing to be bound to any theory, it is believed that up-regulation of tumor-derived Hh ligand post-chemotherapy may confer upon the surviving cell population a dependency upon the Hh pathway that is important for tumor recurrence. These findings are consistent with the observed paracrine cross-talk between the tumor and the surrounding stroma previously shown to be important for Hh signaling (Yauch et al., 2008, Nature 455:406-410).

### Example 12

### Hedgehog Ligand Induction Studies

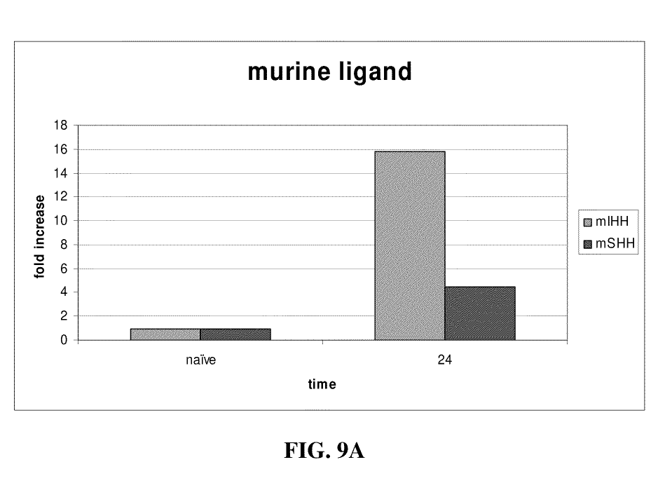

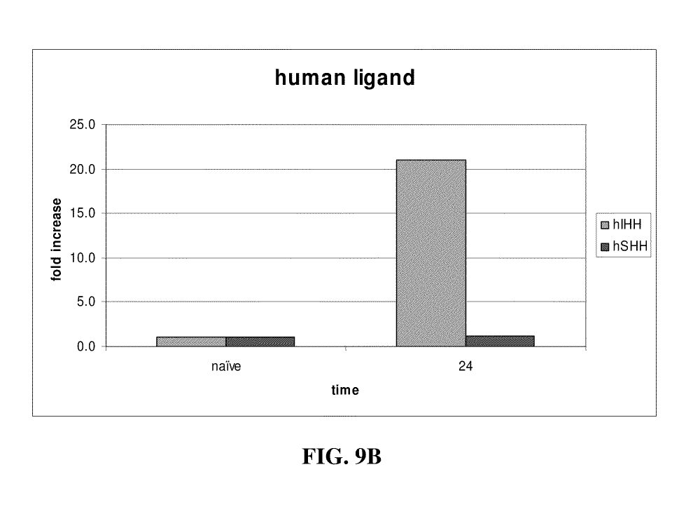

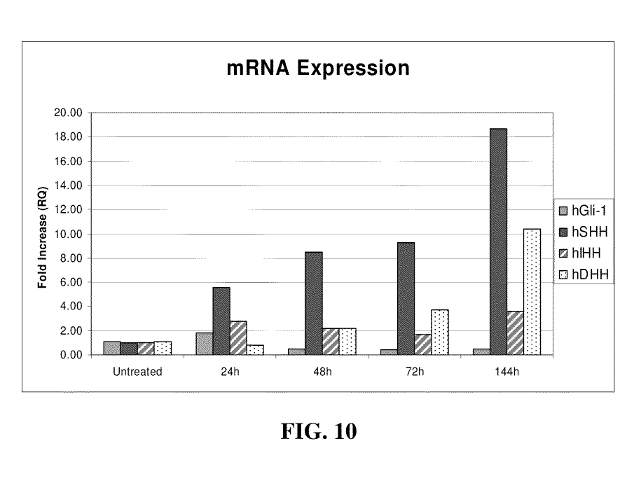

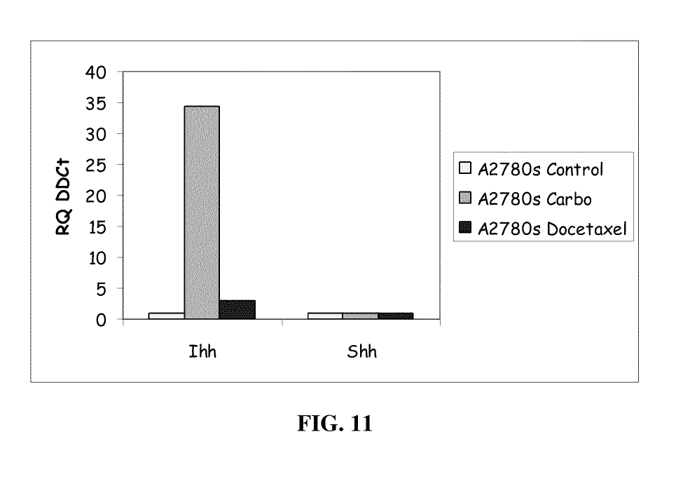

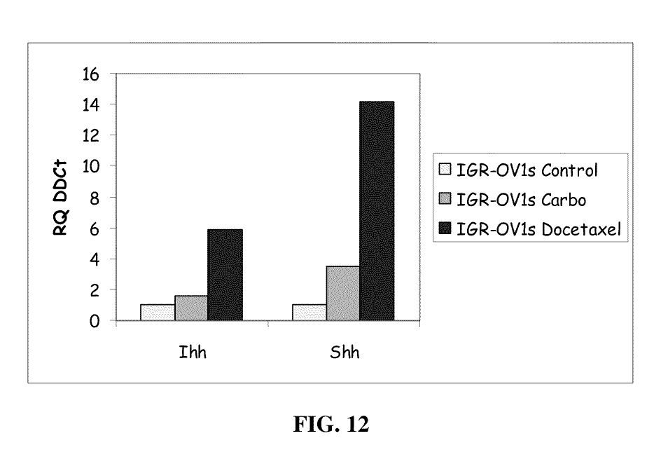

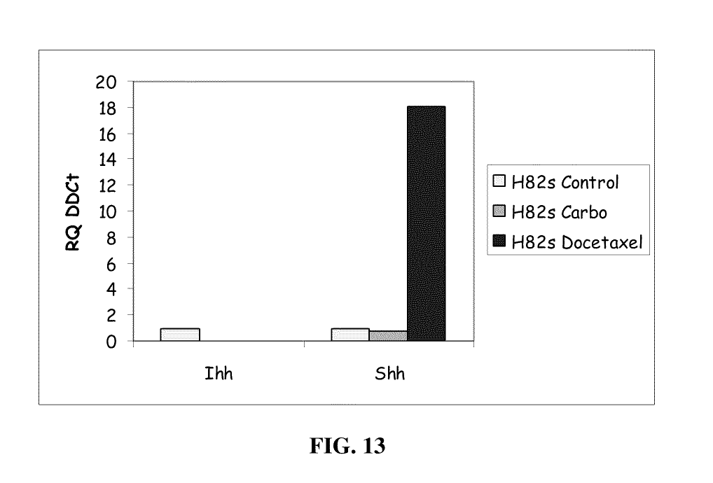

Induction of Hh ligand post chemotherapy was also studied in other cancer tumor models. In vivo, mice bearing UMUC-3 bladder cancer xenografts were treated with 100 mg/kg gemcitabine once-weekly for 4 weeks. Tumors showed increased IHH expression similar to that observed in the LX22 model 24 hours post administration of the final dose (see FIGS. 9A and 9B). In vitro studies showed that in UMUC-3 cells exposed to either doxorubicin or gemcitabine for 12-24 hours, all 3 Hh ligands (Sonic, Indian and Desert) were up-regulated (see doxorubicin data in FIG. 10). Additional in vitro studies showed that IHH expression was increased in A2780 ovarian cancer cells after treatment with carboplatin, while Sonic Hh (SHH) expression was not affected (see FIG. 11), and expression of both IHH and SHH were increased in IGROV-1 cells treated with docetaxel, with SHH being up-regulated to a greater degree (See FIG. 12). Further in vitro studies showed that in small cell lung cancer H82 cells, SHH is up-regulated by docetaxel but not carboplatin, while IHH is not up-regulated by either agent (see FIG. 13).

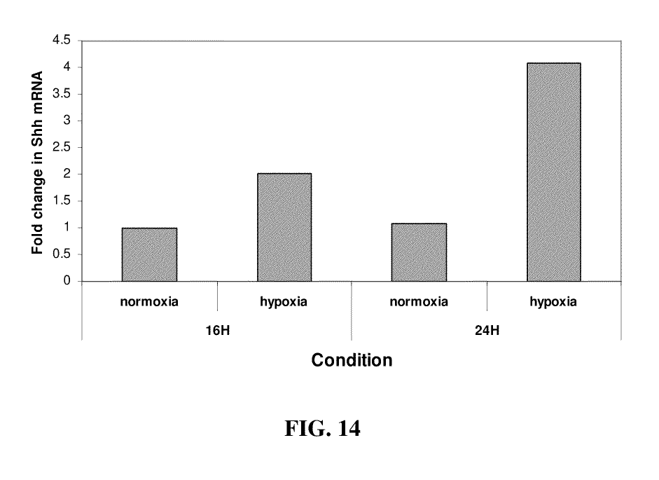

To determine if cellular stresses other than chemotherapy up-regulate Hh ligand expression, UMUC-3 cells were exposed in vitro to various stressors including hypoxia. Compared to normoxic controls, SHH ligand expression was increased at both the RNA and protein level (see FIG. 14).

In summary, multiple tumor types exhibit up-regulation of Hh ligands post chemotherapy. The type of Hh ligand that is up-regulated (i.e., Sonic, Indian and/or Desert) and the degree of up-regulation vary depending upon the tumor type and the chemotherapeutic agent. Without wishing to be bound to any theory, these results suggest that stress (including chemotherapy) induces Hedgehog ligand production in tumor cells as a protective or survival mechanism. The results further suggest that a surviving sub-population may be dependant upon the Hh pathway and thus may be susceptible to Hh pathway inhibition. Taken together, these results indicate that Hedgehog inhibition may increase relapse free survival in clinical indications (such as small cell lung cancer, non-small cell lung cancer, bladder cancer, colon cancer, or ovarian cancer) that are initially chemo-responsive but eventually relapse.

Those skilled in the art will recognize, or be able to ascertain using no more than routine experimentation, many equivalents to the specific embodiments of the invention described herein. Such equivalents are intended to be encompassed by the following claims.

1. A method of extending relapse free survival in a cancer patient who is undergoing treatment with a chemotherapeutic, the method comprising administering a therapeutically effective amount of a hedgehog inhibitor to the patient.

2. The method of claim 1, wherein the hedgehog inhibitor is administered concurrently with the chemotherapeutic.

3. The method of claim 2, wherein administration of the hedgehog inhibitor continues after treatment with the chemotherapeutic has ceased.

4. The method of claim 1, wherein the hedgehog inhibitor is administered after treatment with the chemotherapeutic has ceased.

5. The method of claim 1, wherein the cancer is selected from lung cancer, bladder cancer, ovarian cancer, colon cancer, breast cancer, prostate cancer, multiple myeloma, acute myelogenous leukemia, and chronic myelogenous leukemia.

6. The method of claim 5, wherein the lung cancer is selected from small cell lung cancer and non-small cell lung cancer.

7. The method of claim 1, wherein the cancer is small cell lung cancer and the chemotherapeutic is selected from etoposide, carboplatin, cisplatin, irinotecan, topotecan, gemcitabine, radiation therapy, and combinations thereof.

8. The method of claim 1, wherein the cancer is non-small cell lung cancer and the chemotherapeutic is selected from vinorelbine, cisplatin, docetaxel, pemetrexed, etoposide, gemcitabine, carboplatin, bevacizumab, gefitinib, erlotinib, cetuximab, radiation therapy, and combinations thereof.

9. The method of claim 1, wherein the cancer is bladder cancer and the chemotherapeutic is selected from gemcitabine, cisplatin, methotrexate, vinblastin, doxorubicin, paclitaxel, docetaxel, pemetrexed, mitomycin C, 5-fluorouracil, radiation therapy, and combinations thereof.

10. The method of claim 1, wherein the cancer is ovarian cancer and the chemotherapeutic is selected from paclitaxel, docetaxel, carboplatin, gemcitabine, doxorubicin, topotecan, cisplatin, irinotecan, bevacizumab, radiation therapy, and combinations thereof.

11. The method of claim 1, wherein the cancer is colon cancer and the chemotherapeutic is selected from paclitaxel, 5-fluorouracil, leucovorin, irinotecan, oxaliplatin, capecitabine, bevacizumab, cetuximab, panitumumab, radiation therapy, and combinations thereof.

12. The method of claim 1, wherein the hedgehog inhibitor comprises a compound of formula I: 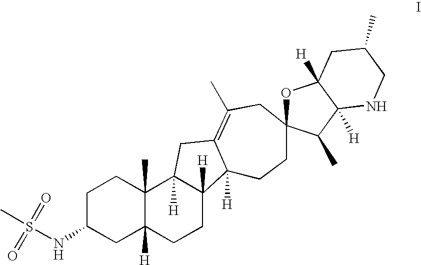
or a pharmaceutically acceptable salt thereof.

13. The method of claim 12, wherein the pharmaceutically acceptable salt is a hydrochloride salt.

14. The method of claim 1, wherein the hedgehog inhibitor is administered as a pharmaceutical composition comprising the hedgehog inhibitor and a pharmaceutically acceptable excipient.

15. A method of extending relapse free survival in a cancer patient who had previously been treated with a chemotherapeutic, the method comprising administering a therapeutically effective amount of a hedgehog inhibitor to the patient after treatment with the chemotherapeutic has ceased.

16. The method of claim 15, wherein the cancer is selected from lung cancer, bladder cancer, ovarian cancer, colon cancer, acute myelogenous leukemia, and chronic myelogenous leukemia.

17. The method of claim 17, wherein the lung cancer is selected from small cell lung cancer and non-small cell lung cancer.

18. The method of claim 15, wherein the cancer is small cell lung cancer and the chemotherapeutic is selected from etoposide, carboplatin, cisplatin, irinotecan, topotecan, gemcitabine, radiation therapy, and combinations thereof.

19. The method of claim 15, wherein the cancer is non-small cell lung cancer and the chemotherapeutic is selected from vinorelbine, cisplatin, docetaxel, pemetrexed, etoposide, gemcitabine, carboplatin, bevacizumab, gefitinib, erlotinib, cetuximab, radiation therapy, and combinations thereof.

20. The method of claim 15, wherein the cancer is bladder cancer and the chemotherapeutic is selected from gemcitabine, cisplatin, methotrexate, vinblastin, doxorubicin, paclitaxel, docetaxel, pemetrexed, mitomycin C, 5-fluorouracil, radiation therapy, and combinations thereof.

21. The method of claim 15, wherein the cancer is ovarian cancer and the chemotherapeutic is selected from paclitaxel, docetaxel, carboplatin, gemcitabine, doxorubicin, topotecan, cisplatin, irinotecan, bevacizumab, radiation therapy, and combinations thereof.

22. The method of claim 15, wherein the cancer is colon cancer and the chemotherapeutic is selected from paclitaxel, 5-fluorouracil, leucovorin, irinotecan, oxaliplatin, capecitabine, bevacizumab, cetuximab, panitumumab, radiation therapy, and combinations thereof.

23. The method of claim 15, wherein the hedgehog inhibitor comprises a compound of formula I: 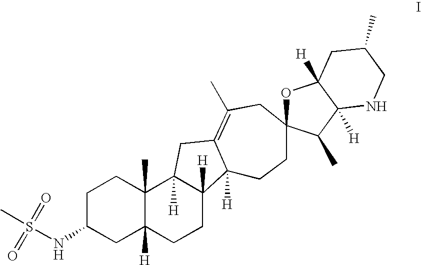
or a pharmaceutically acceptable salt thereof.

24. The method of claim 23, wherein the pharmaceutically acceptable salt is a hydrochloride salt.

25. The method of claim 15, wherein the hedgehog inhibitor is administered as a pharmaceutical composition comprising the hedgehog inhibitor, or a pharmaceutically acceptable salt thereof, and a pharmaceutically acceptable excipient.

26. A method of treating pancreatic cancer, the method comprising administering to a patient in need thereof a therapeutically effective amount of a compound of formula I: 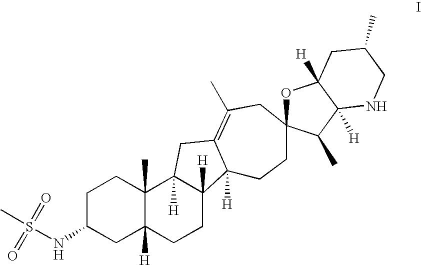
or a pharmaceutically acceptable salt thereof.

27. The method of claim 26, wherein the pharmaceutically acceptable salt is a hydrochloride salt.

28. The method of claim 26, wherein the compound of formula I is administered in combination with a chemotherapeutic.

29. The method of claim 28, wherein the chemotherapeutic is selected from gemcitabine, cisplatin, epirubicin, 5-fluorouracil, and combinations thereof.

30. The method of claim 28, wherein administration of the compound of formula I continues after treatment with the chemotherapeutic has ceased.

31. The method of claim 26, wherein the compound of formula I is administered as a pharmaceutical composition comprising the compound of formula I, or a pharmaceutically acceptable salt thereof, and a pharmaceutically acceptable excipient.

32. A method of treating cancer in a patient wherein the patient is undergoing other cancer therapy, the method comprising detecting elevated hedgehog ligand in the patient and administering a pharmaceutically effective amount of a hedgehog antagonist to the patient.

33. The method of claim 32, wherein the elevated hedgehog ligand is detected in blood, urine, circulating tumor cells, a tumor biopsy or a bone marrow biopsy.

34. The method of claim 32, wherein the step of detecting elevated hedgehog ligand comprises the steps of measuring hedgehog ligand in the patient prior to administration of the other cancer therapy, measuring hedgehog ligand in the patient after administration of the other cancer therapy, and determining if the amount of hedgehog ligand after administration of the other chemotherapy is greater than the amount of hedgehog ligand before administration of the other chemotherapy.

35. The method of claim 32, wherein the other cancer therapy is a chemotherapeutic.

36. A method of treating cancer in a patient, the method comprising identifying one or more chemotherapeutics that elevate hedgehog ligand expression in a tumor, and administering a therapeutically effective amount of the one or more chemotherapeutics that elevate hedgehog ligand expression in the tumor and a therapeutically effective amount of a hedgehog inhibitor.

37. The method of claim 36, wherein the step of identifying one or more chemotherapeutics that elevate hedgehog ligand expression in a tumor comprises the steps of exposing cells from the tumor to one or more chemotherapeutics in vitro, and measuring hedgehog ligand in the cells.
